(19) United States
(12) Patent Application Publication
(10) Pub. No.: US 2002/0077528 A1
(43) Pub. Date: Jun. 20, 2002

(54) Manual instrument for medical and dental diagnostics

(76) Inventor: Hermann Landgraf, Lorsch (DE)

Correspondence Address: Dykema Gossett PLLC, Suite 300 West, 13001 Street, N.W., Washington, DC, 20005-3306, US

(21) Appl. No.: 10/002,006

(22) Filed: Dec. 5, 2001

(30) Foreign Application Priority Data
Dec. 15, 2000 (DE) 100 62 737.4

(51) Int. Cl. A61B 1/04

(52) U.S. Cl. 600/131; 600/112

(57) ABSTRACT

A manual instrument for medical and dental diagnostics having an instrument housing (5). An examination sensor (3) is exchangeably mounted on the proximal end, and an image display device (4) is mounted on its distal end. The image display device (4) has a screen (9) and at least one lighting device (23) with a tension source (22) and light transfer means, as well as a means for image creation is located within the image display device (4). The image display device (4) is swivel-mounted on the instrument housing (5) around a vertical axis (8) extending perpendicularly to the longitudinal symmetrical axis (7) of the manual instrument (2).

### BACKGROUND OF THE INVENTION

This invention is directed to a hand-held instrument or hand piece for medical and dental diagnosis, and more particularly to an instrument capable of sensing and displaying an image for diagnostic purposes.

### DESCRIPTION OF RELATED ART

Hand-held medical instruments are used for multiple examinations in bodily orifices of human beings. Such instruments are used to show the object, that will be examined, on a screen not only to the person which examines, but also to the person which assists and/or to the examined patient himself.

Instruments common in the art have examination sensors with essentially only one electronic optic, which is necessary for the image acquisition and is usually a mini video camera. The image display is a screen that is positioned externally to the person being treated. Although these examination sensors are very small and easy to handle, the presentation of the image display by an external screen is not convenient because the person being treated must turn the visual contact away from the object when viewing the external screen.

It is known in the prior art to arrange the image display device directly on the manual instrument as shown in U.S. Pat. No. 5,879,289. The known manual instrument contains all necessary components, such as a light source with fibre optics that lightens the examination field, means for image creation (CCD) and means for image transmission to the image display device in the manual instrument. The image display device has a mini monitor that is mounted on the housing of the manual instrument. According to one of the embodiments of the present invention, the signals that are created in the image creation device can be transmitted to the externally arranged monitor. As such the same manual instrument can be used in different examination rooms wherein the signals are transmitted by a cable that is connected to the manual instrument and to a central evaluation electronic device. The electronic device is provided with a monitor. The tension supply is optionally made by an integrated tension source or a tension supply, which is arranged externally to the manual instrument.

The mini-monitor is swivel-mounted on the housing by a hinge with a horizontal axis biasly extending to the longitudinal symmetrical axis of the manual instrument. The mini monitor can be swivelled upwardly into the use position from a downwardly swivelled non-use position.

A disadvantage of manual instruments known in the art is that the position of the monitor in the upwardly swivelled state can not be modified. The direction of the view on the monitor is essentially predetermined by the longitudinal symmetrical axis of the manual instrument. Such a rigid arrangement is of a particular disadvantage, if the user wants to communicate with other persons not directly observing the object. For example, the patient is not at all able to observe the image when the sensor is directed to the object.

It is another disadvantage, that the known manual instrument has bad ergonomic handling properties.

Another embodiment of the present invention has the image display device movably fixed to the housing in a manner that allows the image display device to swivel. The image display device of this embodiment can also have a cap formed end piece on the proximal side, which is adjustably mounted into a correspondingly formed end part of the instrument housing.

### SUMMARY OF THE INVENTION

An object of the present device is to create an amelioration and to provide a manual instrument that can be handled in a good ergonomic manner as well by right-handers as by left-handers.

Another object of the present device is to provide an observer who is next to the user of the manual instrument access to the monitor without disrupting the orientation of the sensor in direction of the observed object.

Yet another object of the present invention is to provide an image display device capable of being swivelled around a vertical axis thus allowing the instrument to be adapted to different requirements and needs. In particular, the manual instrument can be used as well by right-handers as by left-handers.

Other objects, advantageous and novel features of the invention will become more apparent from the following detailed description of the invention when taken in conjunction with the accompanying drawings.

### BRIEF DESCRIPTION OF THE DRAWINGS

FIG. 1 is a perspective view of a first embodiment of the manual instrument according to the present invention;

FIG. 2 is a partial top plan view showing a portion in section and the image display device positioned at an angle α from the central axis;

FIG. 3 is a partial side elevation view of a first embodiment of the present invention showing the image display positioned at a right angle from the central axis; and

FIG. 4 is a partial side elevation view of the present invention showing a different housing.

### DETAILED DESCRIPTION OF THE INVENTION

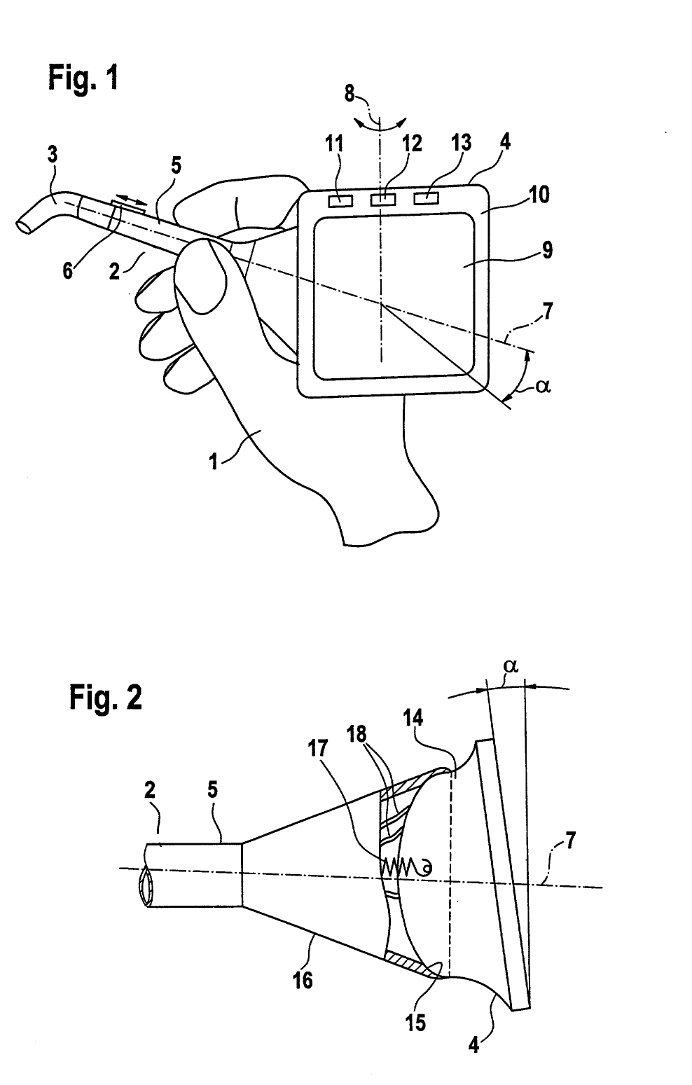

FIG. 1 is a perspective view of a manual instrument 2, which is held in the hand 1 of a user. The instrument 2 has an examination sensor 3 on the proximal side and an image display device 4 on the distal end of the housing 5. The examination sensor 3 is movably mounted on the housing 5 of the manual instrument 2. A pusher 6 is capable of being positioned in the direction of the arrow A. A zoom arranged on the housing 5, which is destinated to amplify or to reduce the image on the image display device 4, can be regulated by the pusher 6.

The image display device 4 is movably mounted in relation to the longitudinal symmetrical axis 7 of the manual instrument 2 and swivels from a median position to both sides. The image display device 4 is movable up to an angle of about 45° around a vertical axis 8, which is perpendicular to the longitudinal symmetrical axis 7. In the embodiment shown in FIG. 1, the manual instrument 2 is held by a right-handed person. The image display device 4 in that figure is swivelled to the left in relation to the observer, so that the observer can observe an image on the screen 9 in an ergonomically advantageous position.

In order to make the observation of the image also possible for a left-handed person, the image display device 4 can be swivelled in the opposite direction by the angle α.

It is especially advantageous that the image display device 4 be capable of swivelling in all directions, not only laterally, but also downwards and upwards. The movable nature of the image display device 4 allows for optimal adaption to different directions of observation, especially for variously positioned observers of the screen 9.

The screen 9 is mounted in a frame 10, on which are arranged several control elements 11, 12 13. Such control elements 11, 12, 13 provide for the modification of the state of the image, such as image amplification, image reduction and image storing.

FIG. 2 shows a top plan view of the distal end of the manual instrument. The image display device 4 has a housing 14 which forms a cap 14, and is arranged in a conically shaped end part 16 of the manual instrument 2. The image display device 4 is not formed in a rotationally symmetrical manner, but is chamfered in the region of the screen. The chamfer can include the angle α.

The image display device 4 has lateral draw springs 17 on the end part 16 that function in such a way that allows the end part 16 to swivel in all directions in the cap guide. In addition, the end part 16 can be rotationally maintained on the front part of the manual instrument 2, so that it can be turned by at least 180° around the longitudinal axis 7. Tube 18 is shown within the housing of the image display 4.

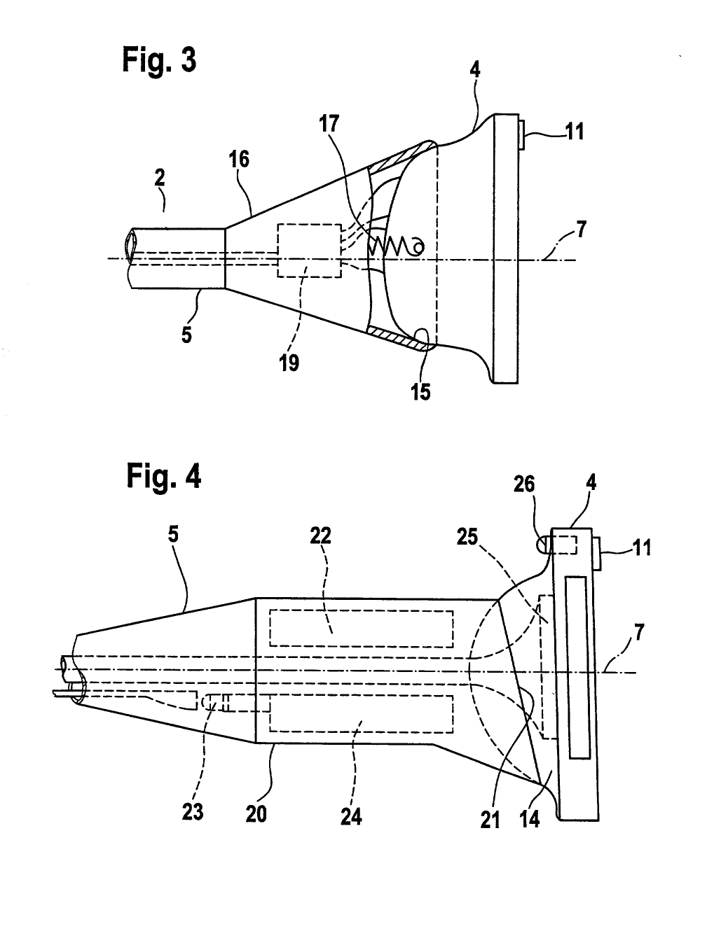

FIG. 3 shows a variation of the first embodiment wherein the image display device 4 is formed symmetrically about the central axis 7. In this embodiment, it is also possible for the cap guide to swivel the image display device 4 in all directions. The electronics 19 (shown in broken lines) are necessary for the image creation and image processing, and are positioned within the conical end part 16 of the instrument housing 5.

Another embodiment is represented in FIG. 4 and has essentially a cylindrical end part 20 with a chamfer 21. The support on the end part 20 is executed, as described before, by draw springs 17 which are not shown in FIG. 4.

A power supply 22 in form of one or several batteries or accumulators, a light source 23 and electronics 24 for image creation and image processing are positioned in the interior of the cylindrical end part 20. The light transfer for lighting the examination field, and for lighting the image transfer from the object to the image creating device 4 is made in a known manner by means of suited fibre optics, which are not indicated in detail in FIG. 4.

In order to change the battery or the accumulators without problems, the end part 20 can have a shaft with a removable corresponding cover. When slim accumulators 25 are used, they can be positioned in the cap shaped housing 14. With such movable action, the housing 5 of the manual instrument 2 can be formed slimmer than with an arrangement of batteries and accumulators 25 in the end part 16.

To transfer the image to a separate external receptor device (not shown), the manual instrument 2 can have an infrared emitter 26 attached on the housing 4 on the rear side of the image display device. The infrared emitter 26 is connected to the electronics 24, which also has an image memory.

What is claimed is:

1. A manual instrument for medical and dental diagnostics comprising:
an instrument housing;
an examination sensor on the proximal end of said instrument; and
an image display device on the distal end of said instrument, said image display device having a screen and at least one lightening device with a tension source, light transfer means, and means for image creation within the interior of said image display device;
said image display device is movably mounted on the instrument housing and swivels around a vertical axis extending perpendicularly to the longitudinal symmetrical axis of the manual instrument.

2. The manual instrument according to claim 1, wherein:
the image display device is mounted on the instrument housing in a manner so that it can be swivelled to all sides.

3. The manual instrument according to claim 2, wherein:
the image display device has a cap formed end piece on the proximal end, said cap is adjustably mounted into a correspondingly formed end part of the instrument housing.

4. The manual instrument according to claim 3, wherein:
the end part of the instrument housing is conically shaped.

5. The manual instrument according to claim 2, wherein:
the end part of the instrument housing is movably mounted and moves around the longitudinal symmetrical axis of the manual instrument.

6. The manual instrument according to claim 1, wherein:
the end part is chamfered.

7. The manual instrument according to claim 1, wherein:
a power supply is arranged in the cap formed end piece.

8. The manual instrument according to claim 1, wherein:
video creating means are arranged in the cap formed end piece.

9. The manual instrument according to claim 1, wherein:
the image display device comprises an emitter for a wireless data transfer of image informations to 4 reception device that is external to the manual instrument.

10. The manual instrument according to claim 9, wherein:
the emitter is an infrared emitter.

11. The manual instrument according to claim 1, wherein:
the frame of the screen comprises a plurality of control elements for modifying the image display state.

12. The manual instrument according to claim 1, wherein:
the examination sensor is movably mounted.

13. The manual instrument according to claim 10, wherein:
said image display state is at least one of image reduction, image amplification and image storing.
